(19) United States
(12) Patent Application Publication
(10) Pub. No.: US 2025/0296014 A1
(43) Pub. Date: Sep. 25, 2025

(54) AN IMPROVED PROCESS OF PURIFICATION OF PROTEIN

(71) Applicant: Kashiv BioSciences, LLC, Piscataway, NJ (US)

(72) Inventors: Om Narayan, Ahmedabad (IN); Tarun Kumar Gupta, Ahmedabad (IN); Mayankkumar Thakkar, Ahmedabad (IN)

(21) Appl. No.: 19/230,884

(22) Filed: Jun. 6, 2025

Related U.S. Application Data
(63) Continuation of application No. 17/922,734, filed on Nov. 1, 2022.

(30) Foreign Application Priority Data
May 1, 2020 (IN) 202021018737
May 1, 2020 (IN) 202021018752

(51) Int. Cl. B01D 15/36; B01D 15/38; C07K 1/18; C07K 16/06; C07K 16/42

(52) U.S. Cl. CPC B01D 15/363; B01D 15/3809; C07K 1/18; C07K 16/065; C07K 16/4291

(57) ABSTRACT

A process for purification of antibody or fusion protein through anion exchange chromatography to produce an antibody or fusion protein which is substantially free of at least one of the product-related impurities.

### CROSS REFERENCE

This application is a continuation of U.S. application Ser. No. 17/922,734, filed on Nov. 1, 2022, which is a national stage entry of International Patent Application No. PCT/IB2021/053659, filed on May 1, 2021, which claims priority to Indian Patent Application number 202021018752 filed on May 1, 2020, and Indian Patent Application number 202021018737 filed on May 1, 2020.

### FIELD OF THE INVENTION

The present invention is directed to the use of anion exchange chromatography to produce an antibody or fragment thereof which is substantially free of at least one of the product-related impurities.

### BACKGROUND OF THE INVENTION

Monoclonal antibodies as a class of therapeutic molecules are finding an increasing demand in the biotechnology industry for the treatment of diseases. Also, these antibodies are heterogeneous in their biochemical and biophysical properties due to multiple posttranslational modification and degradation events occurs during the production. With the advancements in upstream technologies, the capacity for monoclonal antibody (mAb) production has transformed from a few milligrams to grams per liter. These titers lead to enormous pressure on downstream processes (DSPs), which need to be reworked to achieve higher efficiency and better utilization of available resources. If any of these critical parameters are not defined during the facility design stage, collapse of the process can result, further resulting in commercial loss and delaying entry of the product into the market.

Product and process-related impurities must have remained in the acceptable limit set by regulatory bodies for approval.

In conventional methods of purification, product-related impurities are often removed by cation exchange or multimodal chromatography or HIC while process-related impurities are removed by anion exchange chromatography.

Aggregation is one of the product-related impurity which can take place during protein expression in cell culture, purification in downstream processing, formulation, and/or storage. Protein molecules can aggregate via physical association (primary structure unchanged) or by chemical bond formation. Either of them may induce soluble or insoluble aggregates. Over the past few decades, several researchers have proposed different mechanisms of aggregation including (i) reversible association of the native monomer, (ii) aggregation of conformationally altered monomer, (iii) aggregation of chemically modified product, (iv) nucleation-controlled aggregation, and (v) surface-induced aggregation. AAPS J. 2016 May; 18 (3): 689-702. The presence of inactive and/or partially active species is undesirable because these species have a significantly lower binding capacity to the target compared to the active protein; thus, the presence of inactive and/or partially active species can reduce product efficacy. Further, HMW formation may hinder manufacturing. Acidic species are variants with lower apparent pI are a common product-related impurity that is separated by cation exchange chromatography.

Acidic variants substantially affect the in vitro and in vivo properties of antibodies, product stability, product safety therefore it is very imperative to keep acidic variants in the acceptable range of regulatory body to develop acceptable products.

Acidic variants are similar chemical characteristics to the antibody product molecules of interest, reduction of acidic species is a challenge in monoclonal antibody production.

Accordingly, the present invention provides a method of purifying an antibody or fragment thereof having an isoelectric point (pI) from 7 to 8 by anion exchange chromatography wherein the purified antibody or fragment is obtained in flow-through and substantially free of acidic variant below 15% and aggregates below 0.5%. The present process provides a cost-effective and fast process which may reduce the use of additional column to separate acidic variants.

### SUMMARY OF THE INVENTION

The present invention identified the use of anion exchange chromatography (AEX) to reduce product-related impurity of antibody or fusion protein. In certain embodiment, the AEX is strong anion exchange chromatography.

In an embodiment, the present invention provides a process of purifying an antibody or fusion protein with pI of 7 to 8 from the protein mixture comprising antibody or fusion protein and product-related impurities, the purification process comprising:
a. Loading the protein mixture onto anion exchange resin with suitable buffer at suitable pH selected from pH 7.0 to 7.5;
b. Eluting the protein mixture in a flow-through mode whereby product-related impurities binds to the anion exchange resin;
   wherein the eluted protein mixture obtained in step (b) comprises substantially pure monomer of the antibody or fusion protein and reduces the amount of product-related impurities analyzed by analytical HPLC Analysis.

In certain embodiment, the present invention identified the use of anion exchange chromatography (AEX) to reduce HMW and acidic species of antibody. In certain embodiment, the AEX is strong anion exchange chromatography.

In an embodiment, the present invention provides a process of purifying an antibody or fusion protein with pI of 7 to 8 from the protein mixture comprising an antibody or fusion protein and product-related impurities, the purification process comprising:
a. Purifying the protein mixture through affinity chromatography Protein A or Protein G;
b. Subjecting the protein mixture obtained from affinity chromatography to viral inactivation;
c. Loading the protein mixture obtained from step (b) onto anion exchange resin with suitable buffer at suitable pH selected from pH 7.0 to 7.5;
d. Eluting the protein mixture in a flow-through mode whereby product-related impurities bind to the anion exchange resin;
   wherein the eluted protein mixture obtained in step (d) comprises substantially pure monomer of the antibody or fusion protein and reduce the amount of product-related impurities analyzed by analytical HPLC Analysis.

In another embodiment, the present invention provides a process of purifying an antibody or fusion protein with pI of 7 to 8 from the protein mixture comprising antibody or fusion protein and acidic species or variant thereof, the purification process comprising:
a. Purifying the protein mixture through affinity chromatography Protein A or Protein G;
b. Subjecting the protein mixture obtained from affinity chromatography to viral inactivation;
c. Loading the protein mixture obtained from step (b) onto anion exchange resin with suitable buffer at suitable pH selected from pH 7.0 to 7.5;
d. Eluting the protein mixture in a flow-through mode whereby acidic species or variants of said antibody or fusion protein bind to the anion exchange resin;
   wherein the eluted protein mixture obtained in step (d) comprises substantially pure monomer of the antibody or fusion protein and less than 15% of acidic species or variant analyzed by CEX-HPLC Analysis.

In one aspect of such embodiment, wherein the acidic variant is less than about 14% or less AV, 13% or less AV, 12% or less AV, 11% or less AV, 10% or less AV, 9% or less AV, 8% or less AV, 7% or less AV, 6% or less AV, 5% or less AV, 4.5% or less AV, 4% or less AV, 3% or less AV, 2% or less AV, 1% or less AV.

In another embodiment, the present invention provides a process of purifying an antibody or fusion protein with pI of 7 to 8 from the protein mixture comprising antibody or fusion protein and high molecular weight (HMW) impurity, the purification process comprising:
a. Purifying the protein mixture through affinity chromatography Protein A or Protein G;
b. Subjecting the protein mixture obtained from affinity chromatography to viral inactivation;
c. Loading the protein mixture obtained from step (b) onto anion exchange resin with suitable buffer at suitable pH selected from pH 7.0 to 7.5;
d. Eluting the protein mixture in a flow-through mode whereby HMW impurity binds to the anion exchange resin;
   wherein the eluted protein mixture obtained in step (d) comprises substantially pure monomer of the antibody or fusion protein and less than 0.5% of HMW impurity analyzed by SE-HPLC Analysis.

In one aspect of such embodiment, the process provides the protein mixture has high molecular weight species or HMW is 0.5% or less, about 0.4% or less or 0.3% or less or 0.2% or less or 0.1% or less.

In an embodiment, the present invention provides a process of purifying an antibody capable to bind IgE having pI of 7.3 to 7.6 from the protein mixture comprising antibody and product-related impurities comprises acidic species or variant and high molecular weight (HMW), the purification process comprising:
a. Purifying the protein mixture through affinity chromatography Protein A or Protein G;
b. Subjecting the protein mixture obtained from affinity chromatography to viral inactivation;
c. Loading the protein mixture obtained from step (b) onto anion exchange resin with suitable buffer at suitable pH selected from pH 7.0 to 7.5;
d. Eluting the protein mixture in a flow-through mode whereby product-related impurities acidic variants and HMW binds to the anion exchange resin;
   wherein the eluted protein mixture obtained in step (d) comprises substantially pure monomer of the antibody or fusion protein and reduce amount of acidic variant, and HMW analysed by analytical HPLC Analysis.

In one aspect of such embodiment, the purification process reduces acidic variant at least by 25% preferably by 50% in protein mixture obtained in a flow-through mode of strong anion exchange. In one aspect of such embodiment, the purification process reduces HMW at least by 80% preferably by 90% in protein mixture obtained in a flow-through mode of strong anion exchange.

### BRIEF DESCRIPTION OF DRAWINGS AND TABLES

FIG. 1: depicts the complete chromatogram of the AEX run.

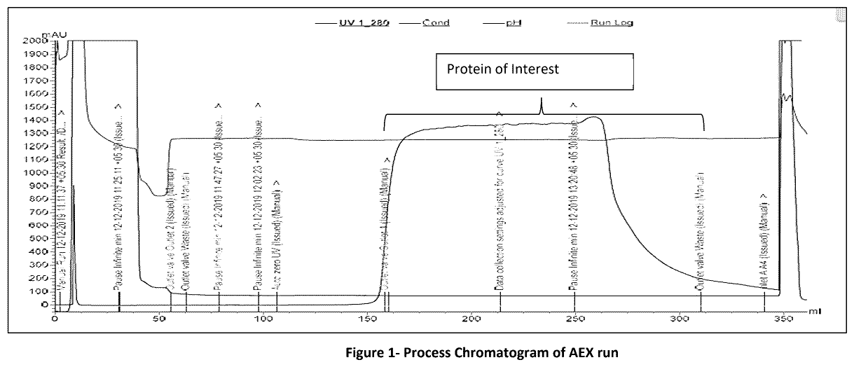

FIG. 2: depicts SE-HPLC Chromatogram of AEX run (Size Variants).

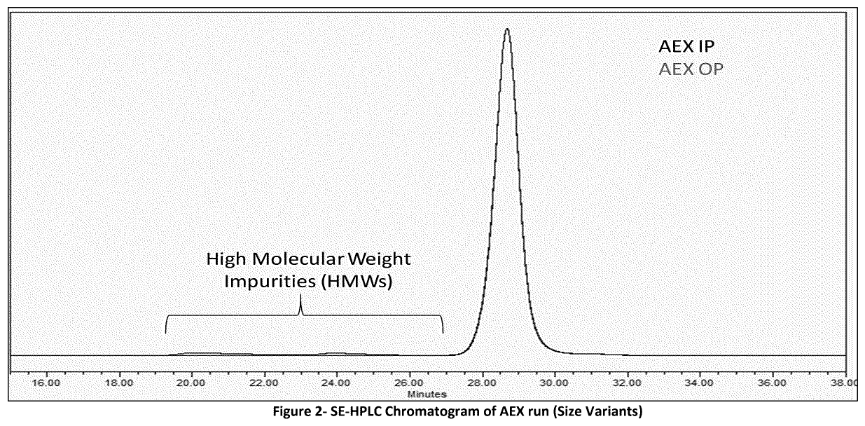

FIG. 3: depicts SE-HPLC Chromatogram of AEX run (Size Variants)—Zoomed in view.

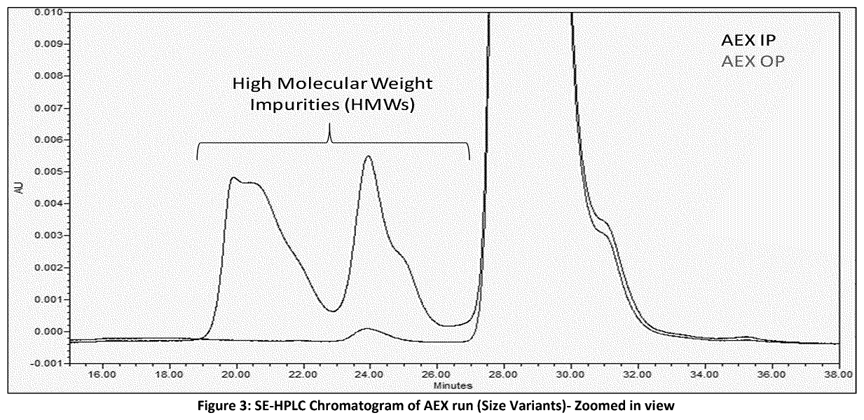

FIG. 4: depicts the comparison of the AEX chromatography input and output charge variant profiles with the help of a Cation Exchange—High-performance Liquid Chromatography overlay.

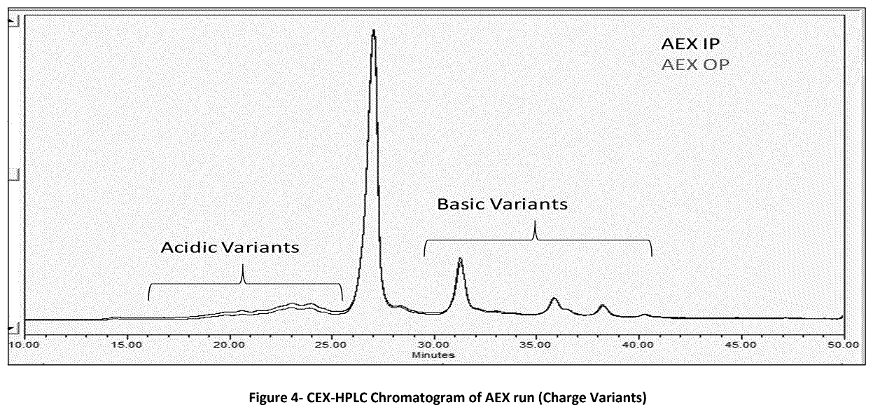

### DETAILED DESCRIPTION OF THE INVENTION

The present invention provides a purification process for removal or reduction of product related impurities by using anion exchange chromatography in flow through mode.

The term “antibody” includes an immunoglobulin molecule comprised of four polypeptide chains, two heavy (H) chains and two light (L) chains inter-connected by disulphide bonds. Each heavy chain is comprised of a heavy chain variable region (abbreviated herein as HCVR or VH) and a heavy chain constant region (CH). The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4.

Omalizumab (Xolair®) is a recombinant DNA-derived humanized IgGIK monoclonal antibody that selectively binds to human immunoglobulin (IgE). The antibody has a molecular weight of approximately 149 kD. Xolair® is produced by a Chinese hamster ovary cell suspension culture in a nutrient medium containing the antibiotic gentamicin. Gentamicin is not detectable in the final product. Xolair® is a sterile, white, preservative-free, lyophilized powder contained in a single-use vial that is reconstituted with Sterile Water for Injection (SWFI), USP, and administered as a subcutaneous (SC) injection. The pI of the Omalizumab is less than 8, preferably about 7.6.

The term used “high molecular weight” or “HMW” is product-related impurities that contribute to the size heterogeneity of antibody products. The formation of HMW species within a therapeutic antibody-drug product as a result of protein aggregation can potentially compromise both drug efficacy and safety (e.g. eliciting unwanted immunogenic response). HMW comprises dimer, trimer, multimers, and aggregates. HMW has considered critical quality attributes that are routinely monitored during drug development and as part of release testing of purified drug products during manufacturing.

The term used “aggregates” are classified based on types of interactions and solubility. Soluble aggregates are invisible particles and cannot be removed with a filter. Insoluble aggregates can be removed by filtration and are often visible to the human eye. Both types of aggregates cause problems in biopharma development. Covalent aggregates arise from the formation of a covalent bond between multiple monomers of a given peptide. Disulfide bond formation of free thiols is a common mechanism for covalent aggregation. Oxidation of tyrosine residues can lead to the formation of bityrosine which often results in aggregation. Reversible protein aggregation typically results from weaker protein interactions they include dimers, trimers, multimers, among others.

As used herein, the terms “acidic variant” or “acidic species” and “AV” refer to the variants of a protein, e.g., an antibody or antigen-binding portion thereof, which are characterized by an overall acidic charge. For example, in monoclonal antibody (mAb) preparations, such acidic species can be detected by various methods, such as ion exchange, for example, WCX HPLC (a weak cation exchange chromatography), or IEF (isoelectric focusing). Acidic variants of antibodies are formed through Chemical and enzymatic modifications such as deamidation and sialylation, respectively, result in an increase in the net negative charge on the antibodies and cause a decrease in p/values, thereby leading to the formation of acidic variants. C-terminal lysine cleavage results in the loss of net positive charge and leads to the acidic variant formation. Another mechanism for generating acidic variants is the formation of various types of covalent adducts, e.g., glycation, where glucose or lactose can react with the primary amine of a lysine residue during manufacturing in glucose-rich culture media or during storage if a reducing sugar is present in the formulation. MAbs. 2010 November-December; 2(6): 613-624.

The term “acidic variant” does not include process-related impurities. The term “process-related impurity,” as used herein, refers to impurities that are present in a composition comprising a protein but are not derived from the protein itself. Process-related impurities include, but are not limited to, host cell proteins (HCPs), host cell nucleic acids, chromatographic materials, protein A contaminant, and media components.

As used herein the term “product-related impurity” refers to the impurity derived from the product of interest for example Acidic variant or HMW.

As used herein the term “Analytical HPLC” refers to CEX-HPLC and SE-HPLC. Charge variants are analyzed by CEX-HPLC and size variants are analyzed by SE-HPLC.

The term “anion exchange chromatography” or “anion exchange column” or “AEX” is a form of “ion-exchange chromatography (IEX)”, which is used to separate molecules based on their net surface charge. Anion exchange chromatography, more specifically, uses a positively charged ion exchange resin with an affinity for molecules having net negative surface charges. Anion exchange chromatography is used both for preparative and analytical purposes and can separate a large range of molecules, from amino acids and nucleotides to large proteins. Here, we focus on the preparative anion exchange chromatography of proteins.

The term “POROS 50 HQ” used herein is a Thermo Scientific™ POROS™ Strong Anion Exchange Resins (POROS AEX resins) are designed for charge-based chromatographic separation of biomolecules including recombinant proteins, monoclonal antibodies. Thermo Scientific™ POROS™ 50 HQ resin is functionalized with quaternized polyethyleneimine groups.

When “strong anion exchange” is used in the flow-through process the equation changes, the impurities are differentiated from the protein of interest, i.e. strong anion exchange is generally known for removal of protein A contaminant, HCP, DNA, or virus in antibody purification. In a flow-through protocol, the sample and equilibration buffer are adjusted to conditions where contaminant molecules will still bind to the resin, but the protein of interest will not (because of the charge). This is achieved by increasing the salt concentration and/or increasing the pH of the buffers to a point below the pI of your molecule of interest.

The present invention surprisingly found the removal of acidic variants and HMW through a strong anion exchange column by performing the column in a flow-through mode wherein the buffer solution pH is 7.0 to 7.3 marginally below the pI of the omalizumab. The optimization of the desired pH of buffer leads to the substantial binding of at least more than 25% to 50% of acidic variant to strong anion exchange. In certain embodiment, the more than 80% to about 95% HMW binds to strong anion exchange.

The present invention provides the purified antibody composition obtained from strong anion exchange wherein the acidic variants are less than 15% preferably less than 12% and HMW less than 0.5% preferably 0.3% which is under the acceptable limit of regulatory bodies.

The present invention is very useful in reducing the burden in downstream processing by avoiding the use of multiple columns. In certain embodiment, the present invention avoids the use of HIC, and multimodal chromatography.

The term “substantially pure antibody” includes an antibody that is substantially free of HMW and acidic variants and specifically binds to IgE. The substantially pure antibody has purity less than about 99% or less than about 98% or less than about 97% or less than about 95% or less than about 92% or less than about 90% or less than about 88% or less than about 85% or less than about 82% less than about 80% or less than about 75% or less than about 70% or less than about 65% or less than about 60% or less than 50%.

As used herein the term “flow-through mode” or “flow-through” refers to a purification process wherein antibody of interest does not bind to chromatography resin. In certain embodiment, the at least 50% antibody of interest does not bind to the chromatographic resin. In certain embodiment, the at least 60% or 70% or 80% antibody of interest does not bind to the chromatographic resin. However, process and product-related impurities bind the chromatographic resin. In certain embodiment, at least 50% of the process and product-related impurities bind to the chromatographic resin. In certain embodiment, at least 60% or 70%, 80%, or 90% process and product-related impurities bind to the chromatographic resin.

As used herein the term “column” or “resin” or “chromatographic resin or chromatographic column” are interchangeable.

The phrase “viral reduction/inactivation”, as used herein, is intended to refer to a decrease in the number of viral particles in a particular sample (“reduction”), as well as a decrease in the activity, for example, but not limited to, the infectivity or ability to replicate, of viral particles in a particular sample (“inactivation”).

The term “comprises” or “comprising” is used in the present description, it does not exclude other elements or steps. For purpose of the present invention, the term “consisting of” is considered to be an optional embodiment, of the term “comprising of”. If hereinafter a group is defined to comprise at least a certain number of embodiments, this is also to be understood to disclose a group which optionally consists only of these embodiments.

As used throughout the specification and in the appended claims, the singular forms “a,” “an,” and “the” include the plural reference unless the context clearly dictates otherwise.

The term “about”, as used herein, is intended to refer to ranges of approximately 10-20% greater than or less than the referenced value. In certain circumstances, one of skill in the art will recognize that, due to the nature of the referenced value, the term “about” can mean more or less than a 10-20% deviation from that value.

In an embodiment, the invention provides a process of purifying an antibody or fusion protein with pI of 7 to 8 from the protein mixture comprising an antibody or fusion protein and product-related impurities, the purification process comprising:
a. Loading the protein mixture onto anion exchange resin with suitable buffer at suitable pH selected from pH 7.0 to 7.5;
b. Eluting the protein mixture in a flow-through mode whereby product-related impurities binds to the anion exchange resin;
   wherein the eluted protein mixture obtained in step (b) comprises substantially pure monomer of the antibody or fusion protein and reduces the amount of product-related impurities analyzed by analytical HPLC Analysis.

Analytical HPLC refers to CEX-HPLC and SE-HPLC. Charge variants are analyzed by CEX-HPLC and size variants are analyzed by SE-HPLC.

In certain embodiment, the product related impurity is acidic variant of the antibody or fusion protein which is reduced by at least 25%, or 40%, or by 50% analyzed by CEX-HPLC. In certain embodiment, the product related impurity is high molecular weight (HMW) impurity of the antibody or fusion protein which is reduced by at least 20%, or 90%, or by 95% analyzed by SE-HPLC.

In an embodiment, the present invention provides a process of purifying an antibody or fusion protein with pI of 7 to 8 from the protein mixture comprising an antibody or fusion protein and product-related impurities, the purification process comprising:
a. Loading the protein mixture onto anion exchange resin with suitable buffer at suitable pH selected from pH 7.0 to 7.5;
b. Eluting the protein mixture in a flow-through mode whereby product-related impurities binds to the anion exchange resin;
   wherein the eluted protein mixture obtained in step (b) comprises substantially pure monomer of the antibody or fusion protein and reduces the amount of product-related impurities analyzed by analytical HPLC Analysis.

In an embodiment, the present invention provides an antibody or fusion protein has pI selected from 7.5, 7.6, 7.7, and 7.8. In the preferred embodiment, the antibody or fusion protein has pI from about 7.4 to about 7.6.

In an embodiment, the buffer used in the anion exchange column has pH selected from 7.0, 7.1, 7.2, 7.3, 7.4, and 7.5 In the preferred embodiment, the buffer has pH from about 7.2 to about 7.4. In the preferred embodiment, the buffer has a pH from about 7.2 to about 7.3.

In an embodiment, the present invention identified the use of anion exchange chromatography (AEX) to reduce product-related impurity selected from HMW and acidic species of antibody.

In an embodiment, the present invention identified the use of anion exchange chromatography (AEX) to reduce HMW and acidic species of antibody. In certain embodiment, the AEX is strong anion exchange chromatography.

In an embodiment, the present invention provides an antibody composition comprising the substantially purified antibody or fragment thereof and a low amount of HMW's.

In an embodiment, the present invention provides an antibody composition comprising a substantially purified antibody or fragment thereof and a low amount of acidic species.

In an embodiment, the present invention provides a process of purifying an antibody or fusion protein with pI of 7 to 8 from the protein mixture comprising an antibody or fusion protein and product-related impurities, the purification process comprising:
a. Purifying the protein mixture through affinity chromatography Protein A or Protein G;
b. Subjecting the protein mixture obtained from affinity chromatography to viral inactivation;
c. Loading the protein mixture obtained from step (b) onto anion exchange resin with suitable buffer at suitable pH selected from pH 7.0 to 7.5;
d. Eluting the protein mixture in a flow-through mode whereby product-related impurities bind to the anion exchange resin;
   wherein the eluted protein mixture obtained in step (d) comprises substantially pure monomer of the antibody or fusion protein and reduces the amount of product-related impurities analyzed by analytical HPLC Analysis.

In an embodiment, the invention provides a process of purifying an antibody or fusion protein with pI of 7 to 8 from the protein mixture comprising antibody or fusion protein and high molecular weight (HMW) impurity, the purification process comprising:
a. Purifying the protein mixture through affinity chromatography Protein A or Protein G;
b. Subjecting the protein mixture obtained from affinity chromatography to viral inactivation;
c. Loading the protein mixture obtained from step (b) onto anion exchange resin with suitable buffer at suitable pH selected from pH 7.0 to 7.5;
d. Eluting the protein mixture in a flow-through mode whereby HMW impurity binds to the anion exchange resin;
   wherein the eluted protein mixture obtained in step (d) comprises substantially pure monomer of the antibody or fusion protein and less than 0.5% of HMW impurity analyzed by SE-HPLC Analysis.

In one aspect of such embodiment, the process provides the protein mixture comprising the HMW is less than 0.5% or less, about 0.4% or less or 0.3% or less or 0.2% or less or 0.1% or less.

In an embodiment, the invention provides a process of purifying a protein mixture comprising an antibody having pI about 7.4 to about 7.6 or aggregate or HMW thereof and one or more variant by using AEX chromatography in flow-through mode by using a buffer at pH about 7.2 to about 7.4, wherein the use of anion exchange chromatography provides a substantially pure an antibody or Variant thereof with Aggregates below 0.5%.

In an embodiment, the invention provides a process of purifying a protein mixture comprising an antibody or aggregate thereof and one or more variants by using AEX chromatography wherein the use of AEX chromatography provides a substantially pure antibody or Variant thereof with Aggregates below 0.2%.

In an embodiment, the invention provides a process of purifying a protein mixture comprising an antibody or aggregate thereof and one or more variants by using AEX chromatography wherein the use of AEX chromatography provides a substantially pure antibody or Variant thereof with Aggregates below 0.1%.

In an embodiment, the invention provides a process of purifying a protein mixture comprising an antibody having pI about 7.4 to about 7.6 or aggregate or HMW thereof and one or more variant by using AEX chromatography in flow-through mode by using a buffer at pH about 7.2 to about 7.4, wherein the use of anion exchange chromatography provides a substantially pure an antibody or Variant thereof with Aggregates below 0.3%.

In an embodiment, the invention provides a process of purifying a protein mixture comprising an antibody having pI about 7.4 to about 7.6 or aggregate or HMW thereof and one or more variant by using AEX chromatography in flow-through mode by using a buffer at pH about 7.2 to about 7.4, wherein the use of anion exchange chromatography provides a substantially pure an antibody or Variant thereof with Aggregates below 0.2%.

In an embodiment, the invention provides a process of purifying a protein mixture comprising an antibody having pI about 7.4 to about 7.6 or aggregate or HMW thereof and one or more variant by using AEX chromatography in flow-through mode by using a buffer at pH about 7.2 to about 7.4, wherein the use of anion exchange chromatography provides a substantially pure an antibody or Variant thereof with Aggregates below 0.1%.

In another embodiment, the present invention provides a process of purifying an antibody or fusion protein with pI of 7 to 8 from the protein mixture comprising antibody or fusion protein and acidic species or variant thereof, the purification process comprising:
a. Purifying the protein mixture through affinity chromatography Protein A or Protein G;
b. Subjecting the protein mixture obtained from affinity chromatography to viral inactivation;
c. Loading the protein mixture obtained from step (b) onto anion exchange resin with suitable buffer at suitable pH selected from pH 7.0 to 7.5;
d. Eluting the protein mixture in a flow-through mode whereby acidic species or variants of said antibody or fusion protein bind to the anion exchange resin;
   wherein the eluted protein mixture obtained in step (d) comprises substantially pure monomer of the antibody or fusion protein and less than 15% of acidic species or variant analyzed by CEX-HPLC Analysis.

In one aspect of such embodiment, the process provides the protein mixture comprising the acidic variant is less than about 14% or less AV, 13% or less AV, 12% or less AV, 11% or less AV, 10% or less AV, 9% or less AV, 8% or less AV, 7% or less AV, 6% or less AV, 5% or less AV, 4.5% or less AV, 4% or less AV, 3% or less AV, 2% or less AV, 1% or less AV.

In an embodiment, the invention provides a process of purifying a protein mixture comprising an antibody or fragment thereof having pI about 7.4 to about 7.6 and acidic variant (AV) by using AEX chromatography in flow-through mode by using a buffer at pH about 7.2 to about 7.4, wherein the use of AEX chromatography provides a substantially pure an antibody or fragment thereof and low amount of AV below 15%.

In an embodiment, the invention provides a process of purifying a protein mixture comprising an antibody or fragment thereof having pI about 7.4 to about 7.6 and acidic variant (AV) by using AEX chromatography in flow-through mode by using a buffer at pH about 7.2 to about 7.4, wherein the use of AEX chromatography provides a substantially pure an antibody or fragment thereof and low amount of AV below 13%.

In an embodiment, the invention provides a process of purifying a protein mixture comprising an antibody or fragment thereof having pI about 7.4 to about 7.6 and acidic variant (AV) by using AEX chromatography in flow-through mode by using a buffer at pH about 7.2 to about 7.4, wherein the use of AEX chromatography provides a substantially pure an antibody or fragment thereof and low amount of AV below 11%.

In an embodiment, the invention provides a process of purifying a protein mixture comprising an antibody or fragment thereof having pI about 7.4 to about 7.6 and acidic variant (AV) by using AEX chromatography in flow-through mode by using a buffer at pH about 7.2 to 7.3 wherein the use of AEX chromatography provides a substantially pure an antibody or fragment thereof and low amount of AV below 10%.

In one aspect of this embodiment, the present invention provides a low AV composition comprising an antibody, or fragment thereof, where the composition comprises about 0.0% to about 15% AV, about 0.0% to about 10% AV, about 0.0% to about 5% AV.

In an embodiment, the invention provides a process of purifying a protein mixture for the separation of acidic species or variants comprising:
a. Purifying the protein mixture through affinity chromatography;
b. Subjecting the protein mixture obtained from affinity chromatography to viral inactivation;
c. Loading the protein mixture obtained from affinity chromatography comprising the antibody has pI about 7.6 capable to bind IgE and acidic variant thereof onto anion exchange column with suitable buffer comprising the pH about 7.2 to about 7.4;
d. Eluting the protein mixture in a flow-through mode whereby a substantial amount of more than 40% acidic species binds to anion exchange resin;
   wherein the eluted protein mixture obtained in step (b) comprises the substantially pure antibody and less than 15% of acidic variant analyzed by CEX-HPLC Analysis.

In an embodiment, the invention provides a process of purifying a protein mixture for the separation of high molecular weight species (HMW) comprising:
a. Purifying the protein mixture through affinity chromatography;
b. Subjecting the protein mixture obtained from affinity chromatography to viral inactivation;
c. Loading the protein mixture obtained from affinity chromatography has pI about 7.6 capable to bind IgE and HMW thereof onto anion exchange column with suitable buffer comprising the pH about 7.2 to about 7.4;
d. Eluting the protein mixture in a flow-through mode whereby more than 50% HMW binds to anion exchange resin;
   wherein the eluted protein mixture obtained in step (d) comprises the substantially pure antibody and less than 0.5% of high molecular weight species analyzed by SE-HPLC Analysis.

In an embodiment, the present invention provides a process wherein the antibody binds to IgE. The antibody is capable to neutralize IgE and thereby reduce or eliminate the symptoms of allergy, asthma, nasal polyps, or urticaria.

In an embodiment, the antibody is Omalizumab. Omalizumab is prepared through recombinant technology and produced through cell culture methods well known in the art.

In another embodiment, the present invention provides a process of purifying an omalizumab antibody from the protein mixture comprising omalizumab and acidic species or variant thereof, the purification process comprising:
a. Purifying the protein mixture through affinity chromatography Protein A or Protein G;
b. Subjecting the protein mixture obtained from affinity chromatography to viral inactivation;
c. Loading the protein mixture obtained from step (b) onto anion exchange resin with suitable buffer at suitable pH selected from pH 7.0 to 7.5;
d. Eluting the protein mixture in a flow-through mode whereby acidic species or variants of said antibody bind to the anion exchange resin;
   wherein the eluted protein mixture obtained in step (d) comprises substantially pure monomer of the antibody and less than 15% of acidic species or variant analyzed by CEX-HPLC Analysis.

In an embodiment, the process provides the protein mixture comprising omalizumab and the acidic variant thereof less than about 14% or less AV, 13% or less AV, 12% or less AV, 11% or less AV, 10% or less AV.

In certain embodiment, the present invention provides a purification process that reduces acidic variant at least by 40% preferably by 50% in protein mixture obtained in the flow-through mode of strong anion exchange.

In an embodiment, the invention provides a process for the purification of antibody or fusion protein from protein mixture comprising protein A or protein G chromatography followed by anion exchange chromatography wherein the anion exchange chromatography reduces at least 25% acidic variant, Wherein the anion exchange is performed in flow-through mode.

In one aspect of such embodiment, the antibody is anti-IgE antibody. In certain embodiment, the anti-IgE antibody is Omalizumab.

In one aspect of such embodiment, the acidic variant is reduced by 40% or by 50% determined by CEX-HPLC.

In an embodiment, the invention provides a process for the purification of antibody or fusion protein from protein mixture comprising protein A or protein G chromatography followed by anion exchange chromatography wherein the anion exchange chromatography reduces at least 20% HMW, wherein the anion exchange is performed in flow-through mode.

In one aspect of such embodiment, the antibody is anti-IgE antibody. In certain embodiment, the anti-IgE antibody is Omalizumab.

In one aspect of such embodiment, the HMW is reduced by 40% or by 50% or by 70% or by 80% or by 90% or by 95% or by 97%.

In an embodiment, the invention provides a pharmaceutical purified composition of Omalizumab comprising product related impurities selected from acidic variant and HMW wherein acidic variant is less than about 9 to about 10% and HMW is less than 0.3% determined by SE-HPLC wherein the purified composition of Omalizumab is obtained from anion exchange chromatography, wherein the anion exchange is performed in flow-through mode.

In another embodiment, the present invention provides a process of purifying an antibody or fusion protein with pI of 7 to 8 from the protein mixture comprising antibody or fusion protein and high molecular weight (HMW) impurity, the purification process comprising:
a. Purifying the protein mixture through affinity chromatography Protein A or Protein G;
b. Subjecting the protein mixture obtained from affinity chromatography to viral inactivation;
c. Loading the protein mixture obtained from step (b) onto anion exchange resin with suitable buffer at suitable pH selected from pH 7.0 to 7.5;
d. Eluting the protein mixture in a flow-through mode whereby HMW impurity binds to the anion exchange resin;
   wherein the eluted protein mixture obtained in step (d) comprises substantially pure monomer of the antibody or fusion protein and less than 0.5% of HMW impurity analyzed by SE-HPLC Analysis.

In one aspect of such embodiment, the purification process reduces the HMW to less than 0.5% or less, about 0.4% or less or 0.3% or less.

In one aspect of such embodiment, the purification process reduces at least 50% or 60% or 70% or 80%, 90%, 91%, 95%, or 97% HMW in the protein mixture.

In certain embodiment, the protein mixture is purified with additional chromatographies like affinity chromatography or cation exchange chromatography or mixed-mode chromatography, or Hydrophobic interaction chromatography, or a combination thereof before treating with anion exchange chromatography.

In certain embodiment, the protein mixture purified by anion exchange chromatography is further purified by affinity chromatography or cation exchange chromatography or mixed-mode chromatography, or Hydrophobic interaction chromatography or combination thereof.

In certain embodiment, the process removes substantial HMW and thereby remove the need of using Hydroxyapatite, HIC, multimodal chromatography.

In certain embodiment, the CHT column is performed after Protein A chromatography.

In certain embodiment, the cation exchange chromatography is performed before anion exchange chromatography.

In certain embodiment, the cation exchange chromatography is performed after protein chromatography and before anion exchange chromatography.

In certain embodiment, the low HMW compositions are produced by subjecting the primary recovery sample to at least one anion exchange separation step. In certain embodiment, the anion exchange step will occur after the above-described affinity chromatography, e.g., Protein A affinity chromatography.

In certain embodiment, the low AV compositions are produced by subjecting the primary recovery sample to at least one anion exchange separation step. In certain embodiment, the anion exchange step will occur after the above-described affinity chromatography, e.g., Protein A affinity chromatography.

In certain embodiment, the use of an anionic exchange material versus a cationic exchange material is based on the local charges of the protein of interest in a given solution. Therefore, it is within the scope of this invention to employ an anionic exchange step before the use of a cationic exchange step, or a cationic exchange step before the use of an anionic exchange step. Furthermore, it is within the scope of this invention to employ only an anionic exchange step, only a cationic exchange step, or any serial combination of the two (including serial combinations of one or both ion exchange steps with the other chromatographic separation technologies described herein).

In an embodiment, the anion exchange chromatography is conditioned where antibody of interest or fragment thereof does not bind to matrix and impurities including but not limited to HMW and AV binds to the matrix and separated from the chromatographic material by washing the material and collecting fractions from the column.

In an embodiment, the anion exchange chromatography resin is selected from Capto Q, DEAE Sepharose fast flow, Fractogel EMD DEAE(M), Toyopearl DEAE-650, Q Sepharose Fast Flow, POROS XQ, POROS 50 HQ, POROS 50 PI, and POROS 50 D. In certain embodiment, the anion exchange chromatography resin is POROS 50 HQ.

In an embodiment, the anion exchange chromatography resin is most preferably selected from POROS 50 HQ.

In an embodiment, the anion exchange resin is a strong anion exchange.

In an embodiment, the product-related impurities are selected from acidic variants and High molecular weight impurities (HMW).

In an embodiment, the present invention provides a process wherein acidic variants and HMW bind to anion exchange resin.

In an embodiment, the present invention provides a process wherein the protein mixture is obtained from affinity chromatography performed prior to anion exchange.

In an embodiment, the equilibration buffer or loading buffer used in the anion exchange column is selected from Sodium Phosphate, Tris-HCl, HEPES, Glycine-NaOH, and Tris-Acetate. In certain embodiment, the equilibration buffer or loading buffer is Tris Acetate.

In an embodiment, the equilibration buffer has a concentration range from about 40 mM to about 60 mM. In a certain embodiment, the loading buffer concentration is about 50 mM.

In an embodiment, the equilibration buffer has a concentration range from about 20 mM to about 60 mM. In a certain embodiment, the loading buffer concentration is about 20 mM.

In an embodiment, the equilibration buffer or loading buffer has a conductivity range from about 1.5 mS/cm to about 3.5 mS/cm. In a certain embodiment, the equilibration or loading buffer conductivity is about 2.6 mS/cm. In the preferred embodiment, the equilibration or loading buffer conductivity is about less than 3 mS/cm. In the preferred embodiment, the equilibration or loading buffer conductivity is about 2 mS/cm.

In an embodiment, the present invention provides a process wherein the antibody or fusion protein has pI selected from 7.5, 7.6, 7.7, and 7.8.

In an embodiment, the present invention provides a process wherein the antibody or fusion protein has pI is preferably selected from 7.6.

In an embodiment, the acidic variants are selected from but not limited to sialylated, deamidated and C-terminal lysine cleavage. In an embodiment, the pH of the equilibration buffer is selected from about 6.5 to about 7.5. In a certain embodiment, the loading buffer pH is about 7.1.

In certain embodiment, the equilibration buffer conductivity is ≤2 mS/cm.

In an embodiment, the pH of the equilibration buffer is selected from about 6.5 to about 7.5. In a certain embodiment, the equilibration buffer pH is about 7.3.

In an embodiment, the loading buffer has a concentration range from about 40 mM to about 60 mM. In a certain embodiment, the loading buffer concentration is about 50 mM.

In an embodiment, the loading buffer has a concentration range from about 10 mM to about 30 mM. In a certain embodiment, the loading buffer concentration is about 20 mM.

In an embodiment, the loading buffer has a conductivity range from about 1.5 mS/cm to about 3.5 mS/cm. In a certain embodiment, the loading buffer conductivity is about 2.6 mS/cm.

In an embodiment, the pH of the loading buffer is selected from about 7.0 to about 7.5. In a certain embodiment, the loading buffer pH is 7.2 to about 7.4.

In certain embodiment, the loading buffer conductivity is about ≤3 mS/cm. In certain embodiment, the loading buffer conductivity is about 2.6 mS/cm.

In an embodiment, the invention provides protein peak Collection criteria selected from the ascending value of about 2.5 AU/cm and ends at a descending value of about 1.5 AU/cm.

In an embodiment, the invention provides protein peak Collection criteria selected from the ascending value of about 1.5 AU/cm and ends at a descending value of about 1.5 AU/cm.

In an embodiment, the invention provides the antibody composition comprising an antibody of interest and about 10% to 12% acidic variant obtained from AEX chromatography wherein the peak collection criteria are selected from about 2.5 AU/cm to about 1.5 AU/cm.

In another embodiment, the invention provides protein peak Collection criteria selected from the ascending value of about 1.5 AU/cm and ends at a descending value of about 1.5 AU/cm.

In an embodiment, the washing buffer is selected from sodium phosphate, Tris-HCl, HEPES, Glycine-NaOH, and Tris-Acetate.

In an embodiment, the washing buffer has a concentration range from about 40 mM to about 60 mM. In certain embodiment, the washing buffer concentration is about 50 mM.

In another embodiment, the washing buffer has a concentration range from about 10 to about 30 mM. In certain embodiment, the washing buffer concentration is about 20 mM.

In an embodiment, the washing buffer has a conductivity range from about 1.5 mS/cm to about 3.5 mS/cm. In the preferred embodiment, the washing buffer conductivity is about 2.6 mS/cm.

In certain embodiment, the washing buffer conductivity is ≤2 mS/cm.

In an embodiment, the pH of the washing buffer is selected from about 7.0 to about 7.5. In a certain embodiment, the washing buffer pH is 7.2 to about 7.4.

In an embodiment, the regeneration buffer is selected from Sodium Phosphate, Tris-HCl, HEPES, Glycine-NaOH, and Tris-Acetate.

In certain embodiment the bound acidic variants and HMWs to the anion exchange resin is eluted through regeneration buffer.

In an embodiment, the regeneration buffer has a concentration range from about 5 mM to about 30 mM. In certain embodiment, the regeneration buffer concentration is about 20 mM.

In an embodiment, the regeneration buffer also contains a salt selected from Sodium Chloride, Potassium chloride, calcium chloride. In certain embodiment, the salt in the regeneration buffer is Sodium Chloride.

In an embodiment, the salt in the regeneration buffer has a concentration range from about 0.5M to about 1.5 M. In certain embodiment, the salt in the regeneration buffer has a concentration of about 1 M.

In an embodiment, the regeneration buffer has conductivity range from about 80 mS/cm to about 90 mS/cm. In certain embodiment, the regeneration buffer conductivity is about 85 mS/cm.

In an embodiment, the regeneration buffer has conductivity range from about 90 mS/cm to about 110 mS/cm. In certain embodiment, the regeneration buffer conductivity is about 100 mS/cm.

In an embodiment, the pH of the regeneration buffer is selected from about 6.5 to about 7.5. In the preferred embodiment, the regeneration buffer pH is about 7.0.

In an embodiment, the pH of the regeneration buffer is selected from about 6.5 to about 7.5. In the preferred embodiment, the regeneration buffer pH is about 7.2. In an embodiment, the elution is performed in a flow-through mode.

In an embodiment, the sanitization buffer is selected from NaOH, Isopropyl alcohol, benzyl alcohol. In certain embodiment, the sanitization buffer is NaOH.

In an embodiment, the sanitization buffer has a concentration range from about 300 mM to about 1500 mM. In certain embodiment, the regeneration buffer concentration is about 500 mM.

In an embodiment, the loading is performed for at least about 5 CVs or more in a certain embodiment, the loading is performed for about 30 CV's.

In an embodiment, the equilibration is performed for at least about 3 CV's to about 10 CV's. In a certain embodiment, the equilibration is performed for about 5 CV's.

In an embodiment, the equilibration is performed until the equilibration buffer conductivity endpoint is achieved.

In an embodiment, the amount of protein loaded onto the column during loading is selected from less than about 150 g/L, less than about 130 g/L, less than about 120 g/L, less than about 110 g/L, less than about 100 g/L.

In an embodiment, the washing is performed for at least about 5 CV's.

In an embodiment, the washing is performed for at least about 2 CV's.

In an embodiment, the regeneration is performed for at least 2 CV's to about 5 CV's. In a certain embodiment, the regeneration is performed for about 3 CV's.

In an embodiment, the regeneration removes most of the impurities. In the preferred embodiment, the regeneration removes most of the HMW and charged-based impurities like acidic variants.

In an embodiment, the sanitization is performed for at least 2 CV's to about 5 CV's. In a certain embodiment, the sanitization is performed for about 3 CV's.

In an embodiment, the sanitization buffer is held in the column for about 15 minutes to about 60 minutes. In certain embodiment, the sanitization buffer is held in the Colum for about 20 minutes.

In an embodiment, the residence time of the protein in the column during AEX purification has a range from about 2 to about 6 minutes. In a certain embodiment, the residence time of the protein in the column is about 4 minutes.

The present invention provides a following examples for illustrative purpose and its scope should not be considered limited to the following examples.

### Example 1—Purification of Monoclonal Antibody Using Strong Anion Exchange Resin

An Omalizumab monoclonal antibody molecule expressed in the Chinese Hamster Ovary (CHO) cell line is captured using Protein A (Mab Select Sure LX, GE Healthcare) packed in VL 11/250 column.

Eluted protein is further subjected to viral inactivation and neutralization. After neutralization, protein has been filtered by 0.2 μm filter.

Post viral inactivation and neutralization step, eluted protein is further purified using Anion Exchange Chromatography resin (POROS 50 HQ, Thermofisher) packed in C10/20 column. The residence time is 4 min for all the phases. After equilibration with Tris Acetate, pH 7.0-7.3 Neutralized Protein A output is loaded at ≤100 mg/mL of the resin. The Load is diluted with water to meet the (mS/cm) conductivity specification 2.5 mS/cm to 2.7 ms/cm before introducing it into the AEX column.

The AEX step is operated in Flow-through (negative) mode and collection is done from 500 mAU ascending to 300 mAU descending of the peak. The column is washed using Tris Acetate, pH 7.0-7.3. AEX output is analyzed with SE-HPLC, CEX-HPLC for size and charge variants. The experimental design for POROS 50 HQ step is summarized in Table 1.

TABLE 1
Experimental design for POROS 50 HQ

| | | Residence | Column |
|---|---|---|---|
| | | Time | Volume |
| Step | Buffer | (min) | (CV) |
| Sanitization | 0.5N Sodium Hydroxide | 4 | 2-3 CV |
| Equilibration | 50 mM Tris Acetate, pH 7.2- | 4 | 3-4 CV |
| | 7.3 | | |
| Load | 50 mM Tris Acetate, pH 7.2- | 4 | Till loading |
| | 7.3 | | volume |
| Wash | 50 mM Tris Acetate, pH 7.2- | 4 | Till absorbance |
| | 7.3 | | 1.5 AU/cm |
| Sanitization | 0.5N Sodium Hydroxide | 4 | 2-3 CV |
| Storage | 0.1N Sodium Hydroxide | 4 | 2-3 CV |

TABLE 2
SE (Size Exclusion)-HPLC (High-performance
Liquid Chromatography Analysis)
SE-HPLC Analysis

| Sample | Main Peak Purity (%) | HMW (%) |
|---|---|---|
| AEX IP | 94.62 | 4.71 |
| AEX OP | 99.34 | 0.07 |

Table 2 shows 99% purity of the main peak and 98% reduction of HMW which is determined by SE-HPLC.

Table 3 shows the results of Cation Exchange-High-performance Liquid Chromatography comparing the main peak with acidic variants.

CEX-HPLC Analysis

| Sample | Main Peak + K1 + K2 (%) | Acidic Variants (%) |
|---|---|---|
| NPEL/AEX IP | 65.86 | 18.28 |
| AEX OP | 74.11 | 8.24 |

Table 3 shows 74% purity of the main peak and approximately 55% reduction of acidic variants which is determined by CEX-HPLC.

### Example 2—Purification of Monoclonal Antibody Using Strong Anion Exchange Resin (50-Liter Batch)

An Omalizumab monoclonal antibody molecule expressed in the Chinese Hamster Ovary (CHO) cell line is captured using Protein A (Mab Select Sure LX, GE Healthcare) packed in VL 11/250 column.

Eluted protein is further subjected to viral inactivation and neutralization. After neutralization, protein has been filtered by 0.2 μm filter.

Post viral inactivation and neutralization step, eluted protein is further purified using Anion Exchange Chromatography resin (POROS 50 HQ, Thermofisher) packed in Chromatographic 100/250 column. The residence time is 4 min for all the phases. After equilibration with Tris HCl, pH 7.1-7.3 Neutralized Protein A output is loaded at 70 to 145 mg/mL of the resin. The Load is diluted with water to meet the (mS/cm) conductivity specification i. e. ≤2.0 mS/cm before introducing it into the AEX column.

AEX step is operated in Flow-through (negative) mode and collection is done from 2.0 AU/cm 5 ascending to 2.5 AU/cm descending of the peak. The column is washed using Tris HCl, pH 7.1-7.3. AEX output is analyzed with SE-HPLC, CEX-HPLC for size and charge variants. The experimental design for POROS 50 HQ step is summarized in Table 4.

TABLE 4
Experimental design for AEX

| | | Residence | Column |
|---|---|---|---|
| | | Time | Volume |
| Step | Buffer | (min) | (CV) |
| Sanitization | 0.5N Sodium Hydroxide | 4 | 3 CV |
| Charge | 20 mM Tris HCl, 1M | 4 | 5 CV or pH end |
| | NaCl pH 7.2 | | point |
| Equilibration | 20 mM Tris HCl, pH 7.2 | 4 | 5 CV or pH end |
| | | | point |
| Load | AEX Load pH 7.2 | 4 | Till loading |
| | conductivity ≤ 2.0 mS/cm | | volume |
| Chase | 20 mM Tris HCl, pH 7.2 | 4 | 3 CV |
| Strip | 20 mM Tris HCl, 1M | 4 | 3 CV |
| | NaCl pH 7.2 | | |
| Sanitization | 0.5N Sodium Hydroxide | 4 | 3 CV |
| Storage | 0.1N Sodium Hydroxide | 4 | 3 CV |

TABLE 5
SE (Size Exclusion)-HPLC (High-performance
Liquid Chromatography Analysis)
SE-HPLC Analysis

| Sample | Main Peak Purity (%) | HMW (%) |
|---|---|---|
| AEX IP | 95.52 | 3.80 |
| AEX OP | 99.45 | 0.14 |

Table 5 shows 99% purity of the main peak and 96% reduction of HMW which is determined by SE-HPLC.

Table 6: Shows the Results of Cation Exchange-High-Performance Liquid Chromatography Comparing the Main Peak with Acidic Variants.

CEX-HPLC Analysis

| Sample | Main Peak (%) | Acidic Variants (%) |
|---|---|---|
| NPEL/AEX IP | 66.53 | 15.80 |
| AEX OP | 67.47 | 11.78 |

Table 6 shows 67% purity of the main peak and approximately 25% reduction of acidic variants which is determined by CEX-HPLC.

### Example 3—Purification of Monoclonal Antibody Using Strong Anion Exchange (200-Liter Batch)

An Omalizumab monoclonal antibody molecule expressed in the Chinese Hamster Ovary (CHO) cell line is captured using Protein A (Mab Select Sure LX, GE Healthcare) packed in VL 11/250 column.

Eluted protein is further subjected to viral inactivation and neutralization. After neutralization, protein has been filtered by 0.2 μm filter.

Post viral inactivation and neutralization step, eluted protein is further purified using Anion Exchange Chromatography resin (POROS 50 HQ, Thermofisher) packed in Chromatographic 350/250 or 200/350 column. The residence time is 4 min for all the phases. After equilibration with Tris HCl, pH 7.1-7.3 Neutralized Protein A output is loaded at 70 to 145 mg/ml of the resin. The Load is diluted with water to meet the (mS/cm) conductivity specification i. e. ≤2.0 mS/cm before introducing it into the AEX column.

AEX step is operated in Flow-through (negative) mode and collection is done from 2.0 AU/cm ascending to 2.5 AU/cm descending of the peak. The column is washed using Tris HCl, pH 7.1-7.3. AEX output is analyzed with SE-HPLC, CEX-HPLC for size and charge variants. The experimental design for POROS 50 HQ step is summarized in Table 7-9.

TABLE 7
Experimental design for AEX

| | | Residence | Column |
|---|---|---|---|
| | | Time | Volume |
| Step | Buffer | (min) | (CV) |
| Sanitization | 0.5N Sodium Hydroxide | 4 | 3 CV |
| Charge | 20 mM Tris HCl, 1M | 4 | 5 CV or pH end |
| | NaCl pH 7.2 | | point |
| Equilibration | 20 mM Tris HCl, pH 7.2 | 4 | 5 CV or pH end |
| | | | point |
| Load | AEX Load pH 7.2 | 4 | Till loading |
| | conductivity ≤ 2.0 mS/cm | | volume |
| Chase | 20 mM Tris HCl, pH 7.2 | 4 | 3 CV |
| Strip | 20 mM Tris HCl, 1M | 4 | 3 CV |
| | NaCl pH 7.2 | | |
| Sanitization | 0.5N Sodium Hydroxide | 4 | 3 CV |
| Storage | 0.1N Sodium Hydroxide | 4 | 3 CV |

TABLE 8
SE (Size Exclusion)-HPLC (High-performance
Liquid Chromatography Analysis)
SE-HPLC Analysis

| Sample | Main Peak Purity (%) | HMW (%) |
|---|---|---|
| AEX IP | 95.80 | 3.71 |
| AEX OP | 99.30 | 0.32 |

Table 8 shows 99% purity of the main peak and 91% reduction of HMW which is determined by SE-HPLC.

Table 9: Shows the Results of Cation Exchange-High-Performance Liquid Chromatography Comparing the Main Peak with Acidic Variants.

CEX-HPLC Analysis

| Sample | Main Peak (%) | Acidic Variants (%) |
|---|---|---|
| NPEL/AEX IP | 69.34 | 15.90 |
| AEX OP | 74.19 | 8.50 |

Table 9 shows 74% purity of the main peak and approximately 46% reduction of acidic variants which is determined by CEX-HPLC.

1. A pharmaceutical purified composition of anti-IgE antibody comprising product related impurities selected from acidic variant and HMW wherein acidic variant is less than about 15% analyzed by CEX-HPLC and HMW is less than 0.5% analyzed by SE-HPLC wherein the purified composition of anti-IgE antibody is obtained from anion exchange chromatography, wherein the anion exchange is performed in flow-through mode; Wherein anti-IgE antibody is omalizumab.

2. The pharmaceutical purified composition of anti-IgE antibody as claimed in claim 1 wherein acidic variant is about 12%.

3. The pharmaceutical purified composition of anti-IgE antibody as claimed in claim 1 wherein acidic variant is about 11%.

4. The pharmaceutical purified composition of anti-IgE antibody as claimed in claim 1 wherein acidic variant is about 10%

5. The pharmaceutical purified composition of anti-IgE antibody as claimed in claim 1 wherein HMW is less than 0.4%.

6. The pharmaceutical purified composition of anti-IgE antibody as claimed in claim 1 wherein HMW is less than 0.3%.

7. The pharmaceutical purified composition of anti-IgE antibody as claimed in claim 1 wherein HMW is less than 0.2%.

8. The pharmaceutical purified composition of anti-IgE antibody as claimed in claim 1 wherein HMW is less than 0.1%.

9. The pharmaceutical purified composition of anti-IgE antibody as claimed in claim 1 wherein the omalizumab is substantially pure having purity more than 90%.

10. The pharmaceutical purified composition of anti-IgE antibody as claimed in claim 1 wherein the omalizumab is substantially pure having purity more than 94%.

11. The pharmaceutical purified composition of anti-IgE antibody as claimed in claim 1 wherein the omalizumab is substantially pure having purity more than 98%.

12. The pharmaceutical purified composition of anti-IgE antibody as claimed in claim 1 wherein the anion exchange is POROS 50 HQ which is strong anion exchange.

13. The pharmaceutical purified composition of anti-IgE antibody as claimed in claim 1, wherein the antibody or fusion protein has pI selected from 7.5, 7.6, 7.7, and 7.9.

14. The pharmaceutical purified composition of anti-IgE antibody as claimed in claim 1, wherein the antibody or fusion protein has a pI of about 7.6.

15. The process for obtaining pharmaceutical purified composition of anti-IgE antibody as claimed in claim 1 comprising:
a) loading the protein mixture onto anion exchange resin with suitable buffer at suitable pH selected from pH 7.0 to 7.5;
b) separating the bound impurities from chromatographic material by washing the material;
c) collecting the fractions from the column.

16. The process as claimed in claim 14, wherein the buffer has pH selected from 7.0, 7.1, 7.2, 7.3, 7.4, and 7.5.

17. The process as claimed in claim 14, wherein the buffer has a pH of about 7.2 or about 7.4.

18. The process as claimed in claim 14, wherein the buffer has a pH of about 7.2 or about 7.3.

19. The process as claimed in claim 14, wherein purified anti IgE antibody is obtained through peak collection in anion exchange chromatography wherein the peak collection is performed from 2.5 AU/cm to about 1.5 AU/cm.

20. The process as claimed in claim 14, wherein purified anti IgE antibody is obtained through peak collection in anion exchange chromatography wherein the protein peak collection criteria selected from the ascending value of about 1.5 AU/cm and ends at a descending value of about 1.5 AU/cm.
